(19) United States
(12) Patent Application Publication
(10) Pub. No.: US 2022/0299509 A1
(43) Pub. Date: Sep. 22, 2022

(54) APPARATUSES, METHODS, COMPONENTS, AND TEST KITS FOR RAPID DIAGNOSTIC TESTS

(71) Applicant: Detect, Inc., Guilford, CT (US)

(72) Inventors: Matthew Dyer, Franklin, IN (US); Todd Roswech, Ivoryton, CT (US); Isaac Bean, Colorado Springs, CO (US)

(21) Appl. No.: 17/700,350

(22) Filed: Mar. 21, 2022

Related U.S. Application Data
(60) Provisional application No. 63/164,179, filed on Mar. 22, 2021.

(51) Int. Cl. G01N 33/543; B01L 3/00; B01L 7/00

(52) U.S. Cl. CPC G01N 33/54388; B01L 3/5029; B01L 7/00; B01L 2300/0832; B01L 2300/0825; B01L 2200/16; B01L 2300/044

(57) ABSTRACT

A rapid diagnostic test apparatus includes: a housing; a sample chamber movably coupled to the housing; and a housing blister compartment arranged in the housing and configured to rupture when the housing is moved relative to the sample chamber from a first position to a second position. The sample chamber may include a vial portion. A vial blister compartment may seal an opening of the vial portion. The sample chamber may include a tube portion disposed in the housing. The vial portion may extend from the housing. The sample chamber may be rotatably coupled to the housing. The housing blister compartment may rupture when the housing is rotated relative to the sample chamber from the first position to the second position. The housing blister compartment may contain a fluid that may be released into the housing when in the second position.

### CROSS-REFERENCE TO RELATED APPLICATIONS

The present application claims the benefit of priority of U.S. Provisional Application No. 63/164,179 filed Mar. 22, 2021, entitled “APPARATUSES, METHODS, COMPONENTS, AND TEST KITS FOR RAPID DIAGNOSTIC TESTS” (Attorney Docket No. H0966.70047US00), the entire contents of which is incorporated by reference herein.

### FIELD

The technology of the present invention relates generally to equipment and methods for rapid diagnostic tests. More specifically, aspects of the technology of the present invention relate to apparatuses, methods, components, and test kits associated with rapid diagnostic tests for detecting the presence of one or more target nucleic-acid sequence(s).

### BACKGROUND

The ability to rapidly diagnose diseases—particularly highly communicable infectious diseases—is critical to preserving human health through early detection and containment of the infectious diseases until reliable preventive measures (e.g., vaccines) and/or medicinal treatments or cures are developed. Rapid testing is of vital to determining infected individuals quickly and minimizing their interactions with others, in order to minimize the spread of the diseases. As one example, the high level of contagiousness, the high mortality rate, and the lack of an early treatment or a vaccine for the coronavirus disease 2019 (COVID-19) have resulted in a pandemic that has already infected millions and killed hundreds of thousands of people. The existence of rapid, accurate diagnostic tests, useable for detecting COVID-19 as well as other diseases, could allow individuals infected with a disease to be quickly identified and isolated, which could assist with containment of the disease. In the absence of such diagnostic tests, diseases such as COVID-19 may spread unchecked throughout communities.

### SUMMARY

Provided herein are apparatuses and techniques for performing diagnostic tests useful for detecting one or more pathogens by detecting one or more target nucleic-acid sequences corresponding to the pathogens. The apparatuses and techniques described herein enable a diagnostic test to be self-administrable by a subject to be tested, and the diagnostic test may be performed in a point-of-care (POC) setting, a non-clinical setting, or a home setting by a lay person without specialized equipment and without training in laboratory procedures. In some embodiments, the apparatuses and techniques described herein may enable rapid diagnostic tests to be performed in less than one hour without sacrificing accuracy. That is, in some embodiments through use of, e.g., isothermal amplification methods, the rapid diagnostic tests enabled by the apparatuses and techniques described herein may provide a diagnosis having an accuracy on par with the accuracy of typical PCR tests in less than one hour.

According to an aspect of the present technology, a rapid diagnostic test apparatus is provided. The test apparatus may be comprised of: a housing; a sample chamber movably coupled to the housing; and a housing blister compartment arranged in the housing and configured to rupture when the housing is moved relative to the sample chamber from a first position to a second position.

In an embodiment of this aspect, the sample chamber may be comprised of a vial portion. A vial blister compartment may seal an opening of the vial portion and may be configured to rupture upon application of a rupture force. In an embodiment, the vial blister compartment may be configured to rupture during insertion of the vial portion into a heater, such that contents of the vial blister compartment may enter the vial portion through the opening.

In an embodiment of this aspect, the sample chamber may be comprised of a tube portion disposed in the housing. The vial portion of the sample chamber may extend from the housing.

In an embodiment of this aspect, the sample chamber may be rotatably coupled to the housing. The housing blister compartment may be configured to rupture when the housing is rotated relative to the sample chamber from the first position to the second position. The housing blister compartment may contain a fluid. In the second position, the fluid may be released into the housing.

According to another aspect of the present technology, a rapid diagnostic test apparatus is provided. The test apparatus may be comprised of: a housing; a sample chamber comprised of a vial coupled to the housing; and a first blister compartment sealing an opening of the vial and configured to rupture upon application of a rupture force.

In an embodiment of this aspect, the vial may extend from a first opening in the housing. The first blister compartment may protrude from a surface of the vial. Application of the rupture force may cause the first blister compartment to be in fluid communication with an internal cavity of the sample chamber.

In an embodiment of this aspect, the housing may be rotated relative to the sample chamber from a rest position to a test position. The housing may be comprised of: a first rotary seal located at the first opening in the housing and configured to provide a leak-tight seal between the housing and the external surface of the vial of the sample chamber; and a second rotary seal located at a second opening in the housing and configured to provide a leak-tight seal between the housing and an external surface of a tube of the sample chamber.

In an embodiment of this aspect, a second blister compartment may be disposed in the housing and may be configured to rupture upon application of a second rupture force. A portion of the tube of the sample chamber may be configured to rupture the second blister compartment in the test position.

According to another aspect of the present technology, a rapid diagnostic test apparatus is provided. The test apparatus may be comprised of: a housing; a sample chamber rotatably coupled to the housing; and a first blister compartment containing a first fluid and sealing an opening of the sample chamber, the first blister compartment being configured to rupture upon application of a rupture force.

In an embodiment of this aspect, the sample chamber may be configured to extend through a cavity of the housing, from a first hole in the housing through a second hole in the housing. The sample chamber may be comprised of a vial portion configured to extend outside of the housing at the first hole. The first blister compartment may protrude from an external surface of the vial portion.

In an embodiment, the sample chamber may be rotated relative to the housing from a rest position to a test position. The housing may be comprised of: a first rotary seal located at the first hole in the housing and configured to provide a leak-tight seal between the housing and an external surface of the vial portion of the sample chamber, and a second rotary seal located at the second hole in the housing and configured to provide a leak-tight seal between the housing and an external surface of the tube portion of the sample chamber.

According to another aspect of the present technology, a method of performing a rapid diagnostic test is provided. The method may be comprised of: providing a sample to a sample chamber of a test apparatus; moving a housing of the test apparatus relative to the sample chamber to cause a housing blister compartment to rupture to release a fluid; and allowing the fluid to interact with the sample.

In an embodiment of this aspect, the moving may be comprised of rotating the housing relative to the sample chamber, from a rest position to a test position. The rotating may cause a surface of the sample chamber to contact the housing blister compartment to rupture the housing blister compartment. In the test position, the housing may be in fluid communication with the sample chamber.

According to another aspect of the present technology, a method of making a rapid diagnostic test apparatus is provided. The method may be comprised of: coupling a housing to a sample chamber such that the housing and the sample chamber are rotatable relative to each other; and providing a blister compartment on the housing. The sample chamber and the housing may be coupled by at least one rotary seal.

In an embodiment of this aspect, the housing may be rotated relative to the sample chamber from a rest position to a test position. In the test position, a surface of the sample chamber may exert a rupture force on the blister compartment.

In another aspect of the present technology, a rapid diagnostic test kit is provided. The test kit may be comprised of: a rapid diagnostic test apparatus comprised of: a housing, a sample chamber coupled to the housing, and a housing blister compartment arranged in the housing and configured to rupture when the housing is moved relative to the sample chamber from a first position to a second position; and a reagent configured to interact with a sample during a test procedure of the test apparatus.

The foregoing and other aspects, embodiments, and features of the present technology described herein can be more fully understood from the following description in conjunction with the accompanying drawings.

### BRIEF DESCRIPTION OF THE DRAWINGS

A skilled artisan will understand that the accompanying drawings are for illustration purposes only. It should be appreciated that the figures are not necessarily drawn to scale and, in some instances, various aspects of the present technology may be shown exaggerated or enlarged to facilitate an understanding of the invention. Items appearing in multiple figures may be indicated by the same reference numeral in some or all of the figures in which they appear. The drawings are not intended to limit the scope of the present teachings in any way.

FIG. 1A depicts a front perspective view of a test apparatus and a sample swab, according to some embodiments of the present technology;

FIG. 1B depicts a front perspective view of the test apparatus of FIG. 1A, according to some embodiments of the present technology;

FIGS. 1C and 1D depict front perspective views of the test apparatus of FIG. 1A in a rest position and a test position, respectively, according to some embodiments of the present technology;

FIG. 2A depicts a right-side elevational view of a test apparatus in a rest position, according to some embodiments of the present technology;

FIG. 2B depicts a right-side elevational view of the test apparatus of FIG. 2A in the rest position and partially inserted in a heater, according to some embodiments of the present technology;

FIG. 2C depicts a right-side elevational view of the test apparatus of FIG. 2B in the rest position and fully inserted in the heater, according to some embodiments of the present technology;

FIG. 2D depicts a right-side elevational view of the test apparatus of FIG. 2C in a test position in first state, while inserted in the heater, according to some embodiments of the present technology;

FIG. 2E depicts a rear elevational view of the test apparatus of FIG. 2D in the test position in a second state, without showing the heating, according to some embodiments of the present technology;

FIG. 3A depicts a right-side elevational view of a test apparatus in a rest position, according to some embodiments of the present technology;

FIG. 3B depicts a right-side elevational view of the test apparatus of FIG. 3A in a test position, according to some embodiments of the present technology;

FIG. 4A shows a flow chart for a method of using a test apparatus, according to some embodiments of the present technology;

FIG. 4B shows a flow chart for a method of making a test apparatus, according to some embodiments of the present technology;

FIG. 4C shows a flow chart for a method of making a test apparatus, according to some embodiments of the present technology; and

FIG. 4D shows a flow chart for a method of making a test kit, according to some embodiments of the present technology.

### DETAILED DESCRIPTION

### 1. Introduction

As the COVID-19 pandemic has highlighted, there is a critical need for rapid, easy to use, accurate systems and methods for diagnosing diseases—particularly infectious diseases. Although diagnostic tests for various diseases, including COVID-19, are known, such tests often require specialized knowledge of laboratory techniques and/or expensive laboratory equipment.

Diagnostic test equipment and test methods described herein may be safely and easily operated or conducted by untrained individuals. Unlike some prior-art diagnostic testing schemes, some embodiments of the technology described herein may not require knowledge of even basic laboratory techniques (e.g., pipetting) or chemistry or biology. Similarly, some embodiments described herein may not require expensive laboratory equipment (e.g., thermocyclers). In some embodiments, one or more reagent(s) may be contained in solid form (e.g., lyophilized form) and/or in liquid form within a container (e.g., a vial, an ampoule, a frangible container, a blister pack, etc.), such that users may avoid exposure to chemicals that may be potentially harmful, and such that contamination of the reagents by the users may be prevented or easily avoided.

Diagnostic test equipment and test methods described herein also may be highly sensitive and accurate. In some embodiments, the diagnostic test equipment and test methods may be configured to detect one or more target nucleic-acid sequence(s) using nucleic-acid amplification (e.g., an isothermal nucleic-acid amplification method). Through nucleic-acid amplification, the diagnostic equipment and methods may be able to detect accurately the presence of extremely small amounts of a target nucleic acid. In some cases, the diagnostic test equipment and components thereof may be available “over-the-counter” (i.e., without a prescription) for use by consumers. In such cases, untrained consumers may be able to self-administer a diagnostic test or administer the diagnostic test to friends and family members in their own homes or in another location of their choosing. In some cases, the diagnostic test equipment and test methods may be operated and performed by employees or volunteers of an organization (e.g., a school, a medical office, a business). For example, a school (e.g., an elementary school, a high school, a university) may test its students, teachers, and/or administrators; a medical office (e.g., a doctor's office, a dentist's office) may test its patients; and a business may test its employees for one or more particular disease(s). In each case, the diagnostic equipment and methods may be operated and performed by test subjects (e.g., students, teachers, patients, employees) or by designated individuals (e.g., a school nurse, a teacher, a school administrator, a receptionist).

The present disclosure may enable diagnostic tests to be performed rapidly to detect one or more target nucleic-acid sequence(s) (e.g., a nucleic acid sequence of a first pathogen, such as SARS-CoV-2, and/or a nucleic acid sequence of a second pathogen, such as an influenza virus). A diagnostic test system, as described herein, may be self-administrable and may comprise a sample-collecting component (e.g., a swab) and a diagnostic test apparatus. In some embodiments of the present technology, a diagnostic test apparatus may comprise a device that allows reagents to be controllably introduced to a sample to, e.g., enable the reagents to interact with the sample individually in a controlled manner. For example, the device be comprised of a housing that includes one or more controllably burstable compartment(s) (e.g., blister pack(s)), according to various embodiments of the present technology. (The terms “rupturable” and “burstable” may be used interchangeably herein to indicate an object that is configured to open (e.g., tear) by use of an appropriate amount of force.) In some embodiments, a diagnostic test apparatus may comprise reagents used in a test procedure of the apparatus. In some other embodiments, a diagnostic test apparatus may be provided separately from reagents used by the apparatus in a test procedure. The reagents of a test procedure may comprise any one or any combination of: lysis reagent(s), nucleic acid amplification reagent(s), CRISPR/Cas detection reagent(s), diluent fluid(s), buffer fluid(s), etc. In some embodiments, a diagnostic test apparatus may comprise a detection component (e.g., a lateral-flow assay (LFA) vehicle) results of which may be self-readable or may be automatically read by a computer algorithm. In some embodiments, a diagnostic test apparatus may comprise a heater or may be used with a heater provided as a separate component. For example, heating may be performed advantageously to amplify RNA and/or DNA targets in a sample by, e.g., an isothermal amplification technique.

In some embodiments of the present technology, diagnostic test apparatuses described herein may be relatively small. For example, a cartridge of a diagnostic test apparatus may be approximately the size of a deck of cards or smaller. Thus, unlike diagnostic tests that require bulky equipment, diagnostic test equipment according to various aspects of the present technology may be easily transported and/or easily stored in homes, schools, and/or businesses. In some implementations of the present technology, diagnostic test equipment described herein may be transported and/or stored at ambient or room temperature such that no special refrigeration (or heating) is needed. This may be true even when transported and/or stored together with reagents used in the equipment.

In some embodiments of the present technology, any reagent(s) contained within a diagnostic test apparatus may be thermostabilized such that the diagnostic test apparatus together with the reagent(s) may be shelf stable for a relatively long period of time (e.g., at least 1 month, at least 3 months, at least 1 year, at least 5 years).

In some embodiments of the present technology, a diagnostic test apparatus may be comprised of one or more blister pack(s). A blister pack may be comprised of one or more compartment(s) (sometimes referred to as “blister compartment(s)”) in which each blister compartment may be a “blister” of the blister pack, according to some embodiments. In some embodiments, each blister compartment may be comprised of one or more solid(s) and/or one or more liquid(s). For example, the solid(s) may be comprised of one or more solid reagent(s) (e.g., lyophilized, dried, crystallized, air jetted, etc., reagent(s)), and the liquid(s) may be comprised of one or more liquid reagent(s) and/or diluent fluid(s). In some embodiments, a blister compartment may be separated from an adjacent chamber or compartment by a breakable seal (e.g., a frangible seal), which may allow reagent(s) and/or fluid(s) within the blister compartment to be released or delivered into the adjacent chamber in a controlled manner (e.g., via controlled breakage of the seal). In some embodiments, a blister compartment comprising a liquid (e.g., a buffer fluid and/or a diluent fluid) may be separated from a chamber comprising a solid reagent (e.g., a lyophilized amplification reagent) and/or a fluid (e.g., a sample fluid) by a seal (e.g., a frangible seal). In some cases, breakage of the seal may allow contents of the blister compartment to interact with contents of the adjacent chamber.

In some embodiments of the technology described herein, the diagnostic test apparatus may comprise blister compartments configured to rupture during different stages of a test procedure. A user may cause a rupture force to be exerted on each of the blister compartments at various stages of the test procedure. In some embodiments, the blister compartments may be configured to rupture when each is squeezed with sufficient force. In some embodiments, a rupture force may be exerted by the user indirectly, without the user touching any of the blister compartments. For example, a rupture force may be exerted indirectly when the user moves a part of the test apparatus relative to another part of the test apparatus without touching any of the blister compartments. In some embodiments, a rupture force may be exerted indirectly by a structure that contacts the blister compartment when the user moves the test apparatus relative to another object used in the test procedure. In some embodiments, an action for causing one blister compartment to rupture may be different from an action for causing another blister compartment to rupture, thus allowing individual blister compartments to be ruptured selectively with little risk of accidentally rupturing a wrong blister compartment.

### 2. Diagnostic Test Apparatuses and Methods

2.1 Test Apparatuses and Test Kits Utilizing Blister-Related Technology

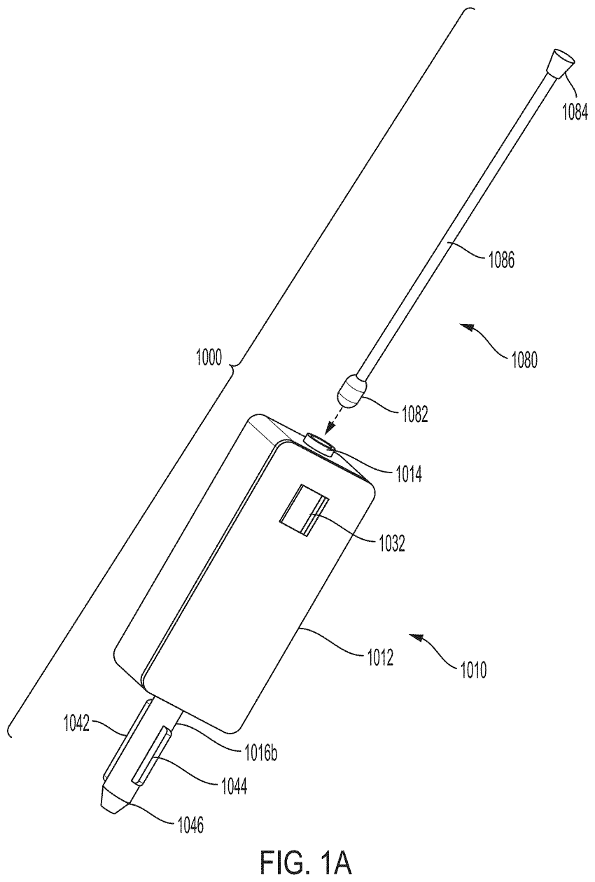

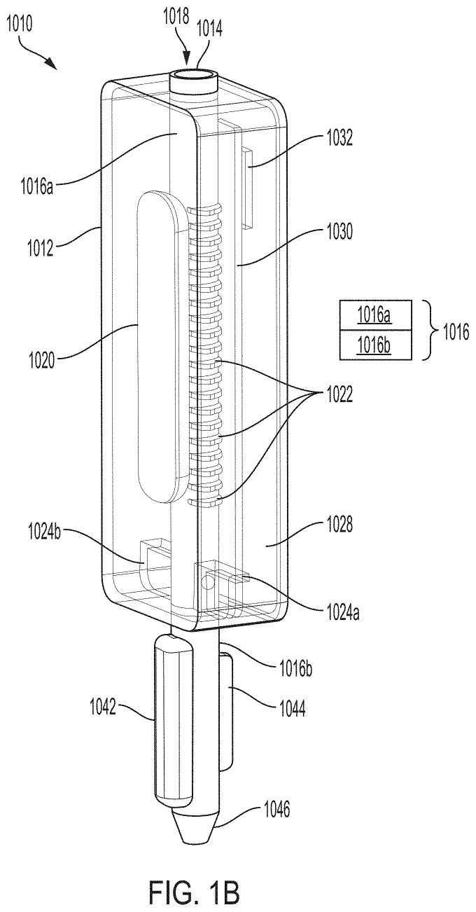

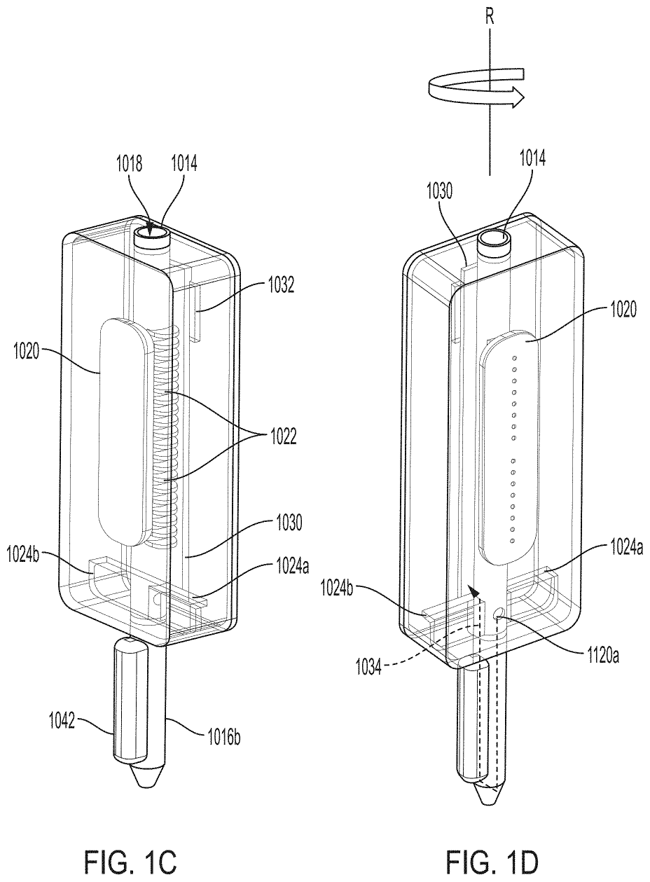

FIG. 1A depicts a perspective view of a diagnostic test kit 1000 comprised of a rapid diagnostic test apparatus 1010 and a sample swab 1080 configured to be inserted in the test apparatus 1010 to provide a sample for a rapid diagnostic test procedure performed by the test apparatus 1010, according to some embodiments of the present technology. In some embodiments, the test kit 1000 may include only the test apparatus 1010. In some embodiments, the test kit 1000 may include the test apparatus 1010 and the necessary reactants and fluids for the test procedure but may not include the sample swab 1080. In some embodiments, the test kit 1000 may include the test apparatus 1010, the necessary reactants and fluids for the test procedure, and the sample swab 1080. The test apparatus 1010 may be comprised of a housing 1012 and a sample chamber 1016. FIG. 1A shows a vial portion 1016b of the sample chamber 1016 extending from the housing 1012.

FIG. 1B depicts a perspective view of the test apparatus 1010 showing the housing 1012 to be clear so that structures internal to the housing 1012 may be seen. In some embodiments of the present technology, the vial portion 1016b of the sample chamber 1016 may be disposed external to the housing 1012 and may extend outwards from a first hole in the housing 1012. The sample chamber 1016 may be comprised of a tube portion 1016a in fluid communication with the vial portion 1016b. The tube portion 1016a may extend from a second hole in the housing 1012, through a housing cavity 1028, to the first hole in the housing 1012. In some embodiments, the first and second holes may be located on opposite sides of the housing 1012, as depicted in FIG. 1B.

In some embodiments of the present technology, the sample chamber 1016 (i.e., the vial portion 1016b and the tube portion 1016a) may be rotated relative to the housing 1012 about an axis of rotation R (see FIG. 1D). In some embodiments, the sample chamber 1016 and the housing 1012 may be rotated relative to each other from a rest position to a test position. In the rest position, the housing cavity 1028 may not be in fluid communication with an internal cavity 1018 of the sample chamber 1016. In the test position, the housing cavity 1028 and the internal cavity 1018 of the sample chamber 1016 may be in fluid communication with each other. The rest position may be, for example, a position at which the apparatus 1010 may be shipped and/or stored. The test position may be, for example, a position at which a testing stage of the test procedure may occur.

In some embodiments of the present technology, the test apparatus 1010 may be comprised of a first rotary seal (not shown) located at the first hole in the housing 1012. The first rotary seal may provide a leak-tight seal between the housing 1012 and the vial portion 1016b of the sample chamber 1016, which may extend outside of the housing cavity 1028. The test apparatus 1010 also may be comprised of a second rotary seal 1014 located at the second hole in the housing 1012. The second rotary seal 1014 may provide a leak-tight seal between the housing 1012 and the tube portion 1016a of the sample chamber 1016. The first and second rotary seals may enable the housing 1012 to be rotated relative to the sample chamber 1016 without leakage of fluid from the housing cavity 1028.

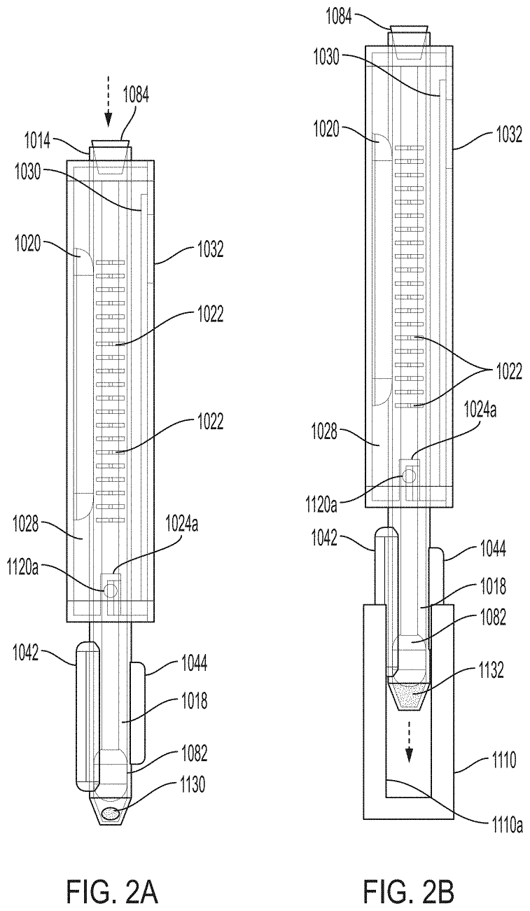

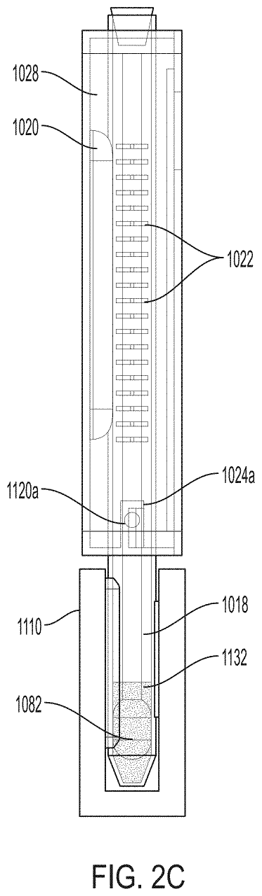

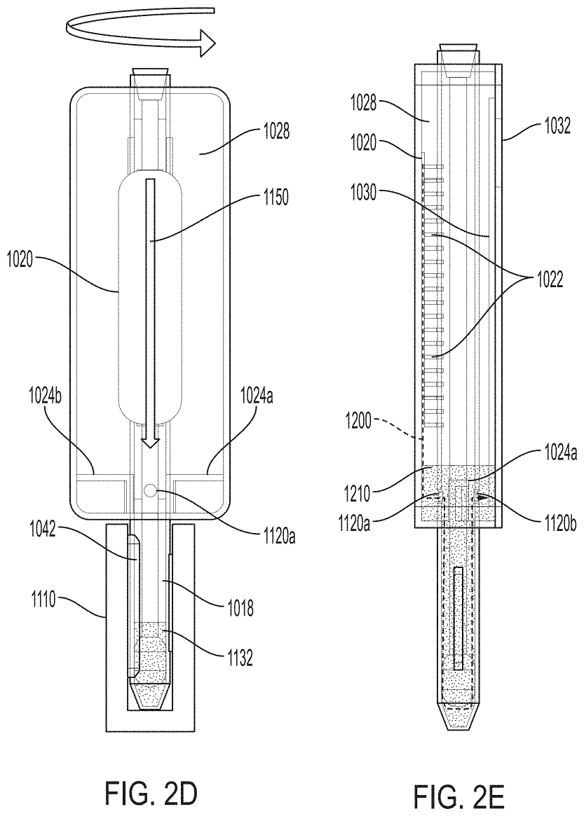

As noted above, the vial portion 1016b and the tube portion 1016a of the sample chamber 1016 may be in fluid communication with each other. In some embodiments of the present technology, the vial portion 1016b may have a closed end 1046 located outside of the housing cavity 1028. The tube portion 1016a may have an open end configured to receive the sample swab 1080 and to enable a swab element 1082 of the sample swab 1080 to be inserted into the internal cavity 1018 of the sample chamber 1016 to the vial portion 1016b. The sample swab 1080 may be comprised of a stem 1086 configured to enable the swab element 1082 to reach the vial portion 1016b when the sample swab 1080 is fully inserted in the sample chamber 1016. In some embodiments, the open end of the tube portion 1016a may be adjacent or nearly adjacent the second rotary seal 1014, and the first rotary seal may be adjacent or near a section of the sample chamber 1016 where the vial portion 1016b meets the tube portion 1016a. In some embodiments, the sample swab 1080 may be configured to remain in the sample chamber 1016 during the test procedure. In such cases, the sample swab 1080 may be comprised of a seal portion 1084 configured to seal the open end of the sample chamber 1016 when the sample swab 1080 is in a fully inserted position in the internal cavity 1018 of the sample chamber 1016. FIG. 2A depicts a right-side elevational view of the apparatus 1010 with the sample swab 1080 in the fully inserted position, according to some embodiments.

In some embodiments of the present technology, the vial portion 1016b and the tube portion 1016a may have been separate parts that have been joined together to form the sample chamber 1016. In some other embodiments, the sample chamber 1016 may be formed of a single structure, with the vial portion 1016b and the tube portion 1016a being separate sections of the single structure. As noted above, the vial portion 1016b and the tube portion 1016a may arranged relative to the housing 1012 such that vial portion 1016b is located outside of the housing cavity 1028 and may extend from the first hole of the housing 1012, and such that the tube portion 1016a may be located inside of the housing cavity 1028 and may extend from the first hole to or through the second rotary seal 1014 at the second hole of the housing 1012.

In some embodiments of the present technology, a blister compartment 1042 may be attached to the vial portion 1016b of the sample chamber 1016 and may surround a hole in the vial portion 1016b. The blister compartment 1042 may protrude outwards from an external surface of the vial portion 1016b. The blister compartment 1042 may contain a fluid therein and may be configured to rupture when a rupture force is applied. For example, when the blister compartment 1042 is squeezed with a force meeting or exceeding the rupture force, the blister compartment 1042 may rupture to enable the fluid therein to flow through the hole in the vial portion 1016b into the internal cavity 1018 the sample chamber 1016. In some embodiments, a lyophilized reagent 1130 may be disposed at a base of the internal cavity 1018 of the sample chamber 1016 (e.g., at or near the closed end 1046 of the vial portion 1016b). The fluid in the blister compartment 1042 may be a buffer fluid configured to activate the reagent 1130 during the test procedure. For example, the reagent 1130 may be an amplification reagent that may form an amplification fluid when in solution with the buffer fluid. When the amplification fluid interacts with a sample carried on the swab element 1082 of the sample swab 1080, a sample fluid 1132 may be formed that may contain amplicons produced from one or more nucleic acid(s) in the sample.

FIG. 2B depicts a right-side side elevational view of the apparatus 1010 during insertion of the vial portion 1016b of the sample compartment 1016 into a heater 1110, according to some embodiments of the present technology. Heating of the sample fluid 1132 may promote production of amplicons, as described herein. In some embodiments, the heating may occur by placing the vial portion 1016b, which is located outside of the housing 1012, into a recess or aperture 1110a of the heater 1110 and activating a heating cycle (e.g., the user may press a button on the heater 1110). In some embodiments, the heating cycle may automatically initiate when the vial portion 1016b is detected to be inserted in the aperture 1110a of the heater 1110. In some embodiments, the aperture 1110a of the heater 1110 may be configured to receive the vial portion 1016b such that the blister compartment 1042 on the vial portion 1016b is ruptured during insertion. For example, during insertion the blister compartment 1042 may be squeezed by a surface of the aperture 1110a of the heater 1110, as schematically shown in FIG. 2B. A force exerted by the user to insert the vial portion 1016b into the aperture 1110a may be sufficient to cause the blister compartment 1042 to be squeezed to the rupture force, thus rupturing the blister compartment 1042 and allowing the fluid to enter the internal cavity 1018 of the sample chamber 1016. When the vial portion 1016b is inserted into the aperture 1110a of the heater 1110, gravitational forces may cause the fluid to flow downwards into the internal cavity 1018 at the vial portion 1016b. The heater 1110 may heat the sample fluid 1132 by heating the vial portion 1016b of the sample chamber 1016.

FIG. 2C depicts a right-side elevational view of the apparatus 1010 when the vial portion 1016b of the sample compartment 1016 is fully inserted in the aperture 1110a of the heater 1110, according to some embodiments of the present technology. As mentioned above, the fluid in the blister compartment 1042 may be a buffer fluid. In some embodiments, an amount of the buffer fluid may be sufficient such that a level of the buffer fluid in the vial portion 1016b may enable the swab element 1082 of the sample swab 1080 to be immersed, to ensure utilization of most if not all of the sample on the swab element 1082 during interaction of the buffer fluid, the reagent 1130, and the sample to form the sample fluid 1132.

According to some embodiments of the present technology, the vial portion 1016b may not include the blister compartment 1042 but instead may include a rupturable ampoule or capsule (not shown) containing the buffer fluid. In these embodiments, when the swab element 1082 is inserted into the vial portion 1016b of the sample chamber 1016, the swab element 1082 may puncture or crush the rupturable ampoule or capsule to release to buffer fluid. The buffer fluid may interact with the reagent 1130 and the sample on the swab element 1082 to form the sample fluid 1132.

In some embodiments of the present technology, the vial portion 1016b of the sample chamber 1016 may be configured such that the sample chamber 1016 may not be rotated relative to the heater 1110 when the vial portion 1016b is seated in the aperture 1110a of the heater 1110. In some embodiments, the vial portion 1016b may have a shape that fits snugly in the aperture 1110a, such that the vial portion 1016b may not be rotated. For example, the vial portion 1016b may have a non-circular cross-sectional shape, and the aperture 1110a may receive the vial portion 1016b such that there is insufficient clearance or room between the aperture 1110a and the vial portion 1016b for relative rotation to be possible.

In some embodiments of the present technology, the vial portion 1016b may be comprised of an anti-rotation structure 1044 located on the external surface of the vial portion 1016b. For example, the anti-rotation structure 1044 may be a tab 1044 that extends outward from the vial portion 1016b. The aperture 1110a may include a slot portion configured to receive the tab 1044 of the vial portion 1016b. With such an arrangement, the vial portion 1016b may be inserted into the aperture 1110a only in a single orientation, and the vial portion 1016b (and the sample chamber 1016) may not be rotated relative to the heater 1110. In another example, the anti-rotation structure 1044 may be a pair of tabs (not shown) that extend outward from diametrically opposite sides of the vial portion 1016b. The aperture 1110a may include a pair of slot portions configured to receive the pair of tabs of the vial portion 1016b. With such an arrangement, the vial portion 1016b may be inserted into the aperture 1110a in two different orientations, and the vial portion 1016b (and the sample chamber 1016) may not be rotated relative to the heater 1110 in either of the two different orientations. As will be appreciated, the anti-rotation structure 1044 of the present technology may comprise other shapes and/or arrangements.

FIGS. 1C and 1D show perspective views of the test apparatus 1010 in a rest position and a test position, respectively, according to some embodiments of the present technology. In some embodiments, a blister compartment 1020 may be located in the housing cavity 1028. For example, the blister compartment 1020 may be attached to a cavity wall of the housing cavity 1028 and may protrude outwards from the cavity wall. For convenience, the blister compartment 1020 in the housing cavity 1028 may be referred to as the “second blister compartment” to avoid confusion with the blister compartment 1042 that may be attached to the vial portion 1016b of the sample chamber 1016. In some embodiments, the second blister compartment 1020 may contain a fluid (“second fluid,” for convenience) therein and may be configured to rupture when a rupture force (“second rupture force,” for convenience) is applied. For example, when the second blister compartment 1020 is squeezed with a force meeting or exceeding the second rupture force, the second blister compartment 1020 may rupture to enable the second fluid therein to flow into the housing cavity 1028.

In some embodiments of the present technology, the blister compartments 1042, 1020 may be formed using known blister-pack technology. In some embodiments, the blister compartments 1042, 1020 may be comprised of a metal foil layer, or a polymer layer, or a combination of at least one metal foil layer and at least one polymer layer. In some embodiments, the blister compartments 1042, 1020 may each be comprised of one or more frangible seal(s). Using known blister-pack technology, the blister compartment 1042 may be configured to rupture into the hole in the vial portion 1016b of the sample chamber 1016 and not into the aperture 1110a of the heater 1110.

In some embodiments of the present technology, the user may cause the housing 1012 to rotate relative to the sample chamber 1016, from the rest position to the test position, while the vial portion 1016b of the sample chamber 1016 is seated in the aperture 1110a of the heater 1110. Because the sample chamber 1016 may not be able to rotate relative to the heater 1110 while the vial portion 1016b is seated in the aperture 1110a, the user may use one hand to hold the heater 1110 and the sample chamber 1016 in a fixed position while the other hand is used to twist the housing 1012 to the test position. Alternatively, the user may hold the housing 1012 in a fixed position with one hand while twisting the heater 1110 and the sample chamber 1016 to the test position with the other hand. In another alternative, the user may perform a twisting movement with both hands, to rotate the housing 1012 and the sample chamber 1016 relative to each other.

In some embodiments of the present technology, the tube portion 1016a of the sample compartment 1016 may be comprised of a rupture surface configured to come into contact with and rupture the second blister compartment 1020 when the housing 1012 is moved relative to the sample chamber 1016 to the test position (FIG. 1D) from the rest position (FIG. 1C). In the rest position, the tube portion 1016a may not contact the second blister compartment 1020 or may contact the second blister compartment 1020 with a force less than the second rupture force, such that the second blister compartment 1020 may be intact in the rest position. In the test position, the rupture surface of the tube portion 1016a may squeeze the second blister compartment 1020 with a force that meets or exceeds the second rupture force, causing the second blister compartment 1020 to rupture and the second fluid to flow into the housing cavity 1028.

In some embodiments of the present technology, the rupture surface of the tube portion 1016a of the sample chamber 1016 may be comprised of at least one rib 1022 that may extend outwards from a surface of the tube portion 1016a. In some embodiments, when the housing 1012 is rotated relative to the sample chamber 1016 to the test position, the rib(s) 1022 may rotate into a position that presses against the second blister compartment 1020 with a force meeting or exceeding the second rupture force. As depicted in FIG. 1C, in the rest position the rib(s) 1022 may not face the second blister compartment 1020 to press against the second blister compartment 1020 but instead may oriented at an angle of, e.g., 90° to the second burstable compartment. In contrast, as depicted in FIG. 1D, in the test position the rib(s) 1022 may face the second blister compartment 1020 directly to press against the second blister compartment 1020.

In some embodiments of the present technology, the rupture surface may be comprised of multiple ribs 1022 arranged to contact different parts of the second blister compartment 1020, such that the second rupture force may be exerted on multiple different parts of the second blister compartment 1020. The use of multiple ribs 1022 may help ensure that at least a minimum amount of the second fluid necessary for the test procedure is squeezed out of the second blister compartment 1020 regardless of where the second blister compartment 1020 ruptures. That is, even if the second blister compartment 1020 ruptures to form a hole in an upper region of the second blister compartment 1020 (e.g., closer to the second rotary seal 1014), where gravitational forces may prevent the second fluid from freely flowing out of the second blister compartment 1020, the multiple ribs 1022 of the tube portion 1016a may squeeze a sufficient quantity of the second fluid out of the second blister compartment 1020 and into the housing cavity 1028 for use in the test procedure. In some embodiments, instead of the multiple ribs 1022, the rupture surface may be comprised of an elongate surface configured to squeeze an area of the second blister compartment 1020 to enable a sufficient quantity of the second fluid to flow into the housing cavity 1028. For example, the tube portion 1016a may be comprised of a section having a non-circular lateral cross-section with respect to the axis of rotation R of the sample chamber 1016, such that a part of the tube portion 1016a having a relatively larger radius from the axis of rotation R than another part of the tube portion 1016a may be rotated to the test position to squeeze the second blister compartment 1020. It should be understood that various other configurations may be employed for the rupture surface of the present technology, and the configurations described herein are only examples of the rupture surfaces that may be used.

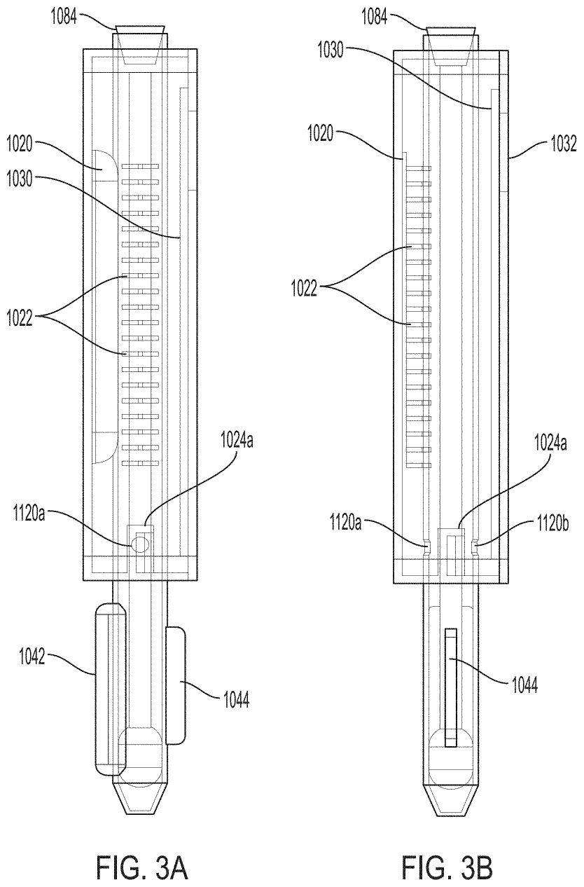

As noted above, FIG. 2C depicts a right-side elevational view of the test apparatus 1010 in the rest position when the vial portion 1016b of the sample chamber 1016 is fully inserted in the aperture 1110a of the heater 1110, according to some embodiments of the present technology. FIG. 2D depicts the test apparatus 1010 of FIG. 2C after the housing 1012 has been rotated relative to the sample chamber 1016 to the test position, according to some embodiments. In FIGS. 2C and 2D, the sample chamber 1016 is depicted in the same orientation to show how the position of the housing 1012 may change. FIG. 3A depicts a right-side elevational view of the apparatus 1010 in the rest position without the heater 1110, and FIG. 3B depicts a rear elevational view of the apparatus 1010 in the test position without the heater 1110. In FIGS. 3A and 3B, the housing 1012 is depicted in the same orientation to show how the position of the sample chamber 1016 may change (the heater 1110 is not shown so that the vial portion 1016b of the sample chamber 1016 may be seen more easily).

In some embodiments of the present technology, the tube portion 1016a of the sample chamber 1016 may be comprised of a plurality of holes 1120a, 1120b, which may form conduits between the housing cavity 1028 and the internal cavity 1018 of the sample chamber 1016. The housing 1012 may be comprised of blockers 1024a, 1024b located at fixed positions in the housing cavity 1028. When the housing 1012 is in the rest position relative to the sample chamber 1016 (see FIGS. 2A-2C), the blockers 1024a, 1024b may block the holes 1120a, 1120b in the tube portion 1016a to prevent movement of fluid between the housing cavity 1028 and the internal cavity 1018 of the sample chamber 1016. That is, in the rest position, fluid communication between the housing cavity 1028 and the internal cavity 1018 of the sample chamber 1016 may be prevented. When the housing 1012 is moved (e.g., rotated) relative to the sample chamber 1016 to the test position, the holes 1120a, 1120b in the tube portion 1016a may be free from blockage by the blockers 1024a, 1024b of the housing 1012 (see FIG. 2D), such that fluid in the housing cavity 1028 may flow into the internal cavity 1018 of the sample chamber 1016 via at least one of the holes 1120a, 1120b in the tube portion 1016a, and such that fluid in the internal cavity 1018 of the sample chamber may 1016 flow into the housing cavity 1028 via at least one of the holes 1120a, 1120b in the tube portion 1016a.

In some embodiments of the present technology, the second fluid in the second blister compartment 1020 may be a diluent fluid configured to dilute the sample fluid 1132. In some embodiments, after the sample fluid 1132 has been formed and heated by the heater 1110, the sample fluid 1132 may be diluted to form a diluted sample fluid 1210 usable on a test vehicle to test for one or more pathogen(s) in the sample fluid 1132, as discussed herein. When the housing 1012 is moved relative to the sample chamber 1016 to the test position, the diluent fluid in the second blister compartment 1020 may flow out of the second blister compartment 1020 into the housing cavity 1028 and toward the holes 1120a, 1120b of the tube portion 1016a, as depicted by an arrow 1150 in FIG. 2D. The diluent fluid in the housing cavity 1028 may flow through the (unblocked) holes 1120a, 1120b into the internal cavity 1018 of the sample chamber 1016 where the diluent fluid may mix with the sample fluid 1132 to form the diluted sample fluid 1210.

A meandering fluid path is represented by an arrow 1034 in FIG. 1D, which shows a front perspective view of the test apparatus 1010 in the test position, and by an arrow 1200 in FIG. 2E, which shows a rear elevational view of the test apparatus 1010 in the test position. As more and more of the diluent fluid flows into the internal cavity 1018 of the sample chamber 1016, a volume of the diluted sample fluid 1210 in the internal cavity 1018 of the sample chamber 1016 may increase. When an amount of the diluted sample fluid 1210 in the internal cavity 1018 is sufficient to reach the holes 1120a, 1120b in the tube portion 1016a, the diluted sample fluid 1210 may enter the housing cavity 1028.

In some embodiments of the present technology, an LFA strip 1030 may be disposed in the housing cavity 1028. For example, as shown by the arrows 1034 and 1200 in FIGS. 1D and 2E, the diluent fluid may flow from the second blister compartment 1020 into the housing cavity 1028 and through the hole 1120a into the internal cavity 1018 of the sample chamber 1016 where the diluent fluid may mix with the sample fluid 1132 to form the diluted sample fluid 1210. In turn, the diluted sample fluid 1210 may flow through the hole 1120b into the housing cavity 1028 where the diluted sample fluid 1210 may contact the LFA strip 1030 and wet at least an end portion of the LFA strip 1030.

As noted above, by gravity the diluent fluid may flow downwards and through at least one of the holes 1120a, 1120b of tube portion 1016a of the sample chamber 1016, according to various embodiments of the present technology. In some embodiments, the end portion of the LFA strip 1030 may be located at a height that is higher than the holes 1120a, 1120b of the tube portion 1016a of the sample chamber 1016, to prevent the end portion of the LFA strip 1030 from contacting the diluent fluid before the diluent fluid mixes with the sample fluid 1132 to form the diluted sample fluid 1210. In some embodiments, the holes 1120a, 1120b may be at the same height. In some other embodiments, the holes 1120a, 1120b may be at different heights. With such an arrangement, the end portion of the LFA strip 1030 may not come into contact with the diluent fluid as the diluent fluid flows downward, and thus in some embodiments the LFA strip 1030 may remain dry or may have minimal contact with fluid until an amount of the diluted sample fluid 1210 in the housing cavity 1028 has reached a level sufficient to touch the end portion of the LFA strip 1030.

In some embodiments of the present technology, the end portion of the LFA strip 1030 may absorb the diluted sample fluid 1210, and the diluted sample fluid 1210 may then travel along the LFA strip 1030 to a test portion of the LFA strip 1030 via capillary action. The test portion may be configured to detect one or more target nucleic-acid sequence(s) corresponding to one or more pathogen(s), as described herein. In some embodiments, the housing 1012 may be comprised of a window 1032 through which at least the test portion of the LFA strip 1030 may be visible, to enable the test portion to be read by a human and/or by a machine.

In some embodiments of the present technology, the LFA strip 1030 may be used to test for a plurality of different pathogens or target nucleic-acid sequences (also referred to as “target nucleic acids” herein) in a single test procedure. In some embodiments, the LFA strip 1030 may provide results that may be read or interpreted in a non-clinical setting by a lay person (e.g., a person not trained in laboratory procedures). The LFA strip 1030 may be comprised of reactants for indicating the presence (or absence) or each of the multiple different target nucleic-acid sequences. In some embodiments, the LFA strip 1030 may be configured to detect two or more target nucleic-acid sequences. In certain cases, the LFA strip 1030 may be comprised of one or more fluid-transporting layer(s), which may be comprised of one or more absorbent material(s) that allow fluid transport (e.g., via capillary action). Non-limiting examples of suitable materials may include polyethersulfone, cellulose, polycarbonate, nitrocellulose, sintered polyethylene, and glass fibers.

2.2 Methods Utilizing Blister-Related Technology

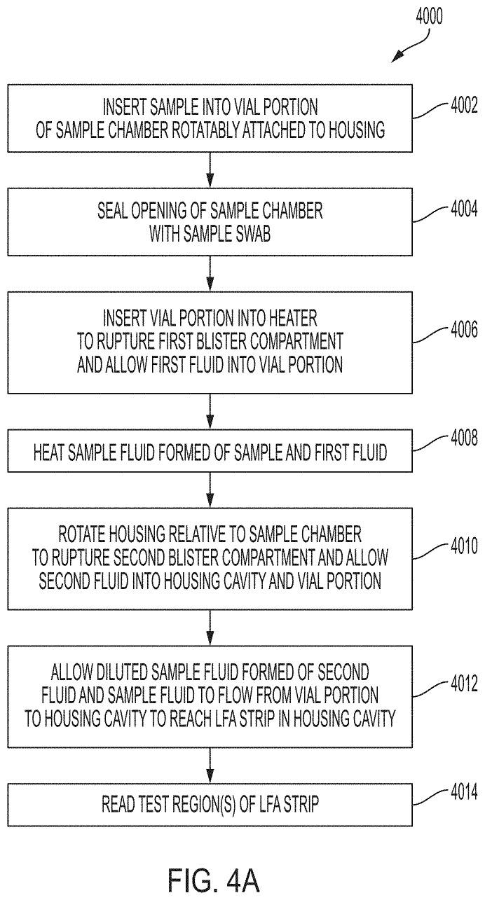

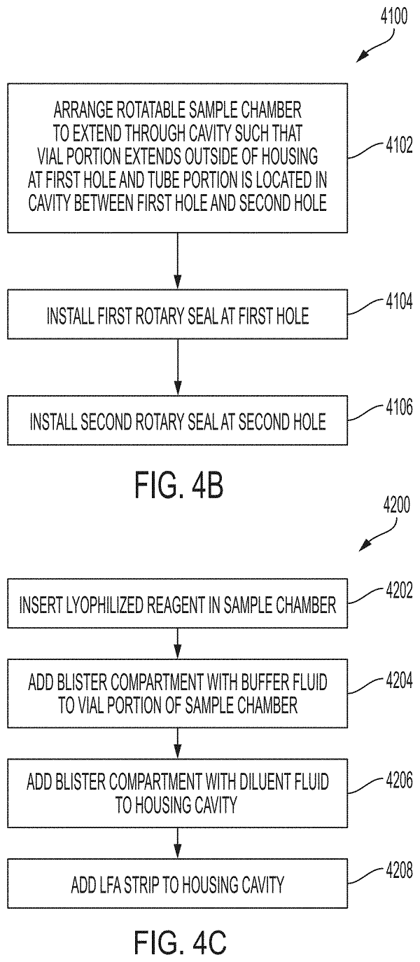

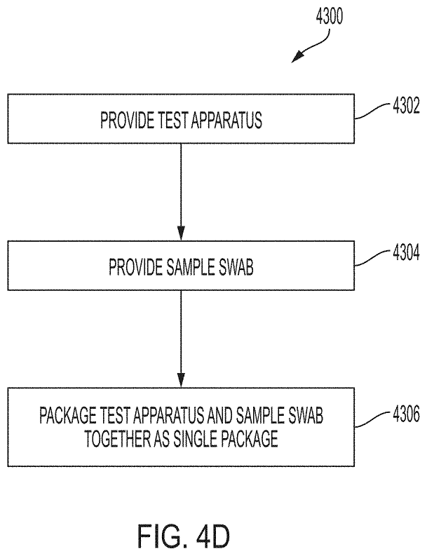

FIG. 4A shows a flow chart for a method 4000 of using a diagnostic test apparatus (e.g., the test apparatus 1010) for a test procedure, according to some embodiments of the present technology. As will be appreciated, acts of the method 4000 need not be performed in the order shown in the flow chart. At act 4002, a sample swab (e.g., the sample swab 1080) may be inserted into an opening of a sample chamber (e.g., the sample chamber 1016) of the apparatus. The sample swab may carry a sample on a swab element of the sample swab. At act 4004, the opening of the sample chamber may be sealed. For example, if the sample swab is to remain in the sample chamber during the test procedure, the opening may be sealed by a seal portion of the sample swab. In another example, the opening may be sealed by, e.g., a cap or a stopper. At act 4006, a first blister compartment (e.g., the blister compartment 1042) may be ruptured to enable a first fluid to enter an internal cavity of the sample chamber from the first blister compartment. For example, the first blister compartment may protrude from a vial portion (e.g., the vial portion 1016b) of the sample chamber, and the first blister compartment may rupture during insertion of the vial portion into an aperture of a heater (e.g., the heater 1110) used to heat the vial portion. In some embodiments, the first fluid may interact with the sample on the swab element and a lyophilized reagent in the internal cavity of the sample chamber to form a sample fluid. At act 4008, the vial portion is heated. For example, heating of the vial portion may in turn heat the sample fluid to promote amplicon production in the sample fluid. At act 4010, a housing (e.g., the housing 1012) of the apparatus may be rotated relative to the sample chamber, to cause a second blister compartment (e.g., the blister compartment 1020) to be ruptured to enable a second fluid to enter a housing cavity of the housing and to flow into the internal cavity of the sample chamber from the housing cavity. In some embodiments, the sample chamber may extend through the housing and may be rotated relative to the housing via rotary seals. For example, the second fluid may be a diluent fluid configured to dilute the sample fluid. At act 4012, the diluted sample fluid may contact a LFA test strip (e.g., the LFA strip 1030) in the housing cavity. For example, the diluted sample fluid may flow from the internal cavity of the sample chamber into the housing cavity to contact the LFA strip. At act 4014, after interaction with the diluted sample fluid, the LFA test strip may be read. For example, one or more test region(s) of the LFA strip may be read by an electronic device (e.g., a smartphone camera), which may be programmed to recognize a particular appearance of the test region(s) to indicate a presence of one or more pathogen(s) in the sample.

FIG. 4B shows a flow chart for a method 4100 of making a diagnostic test apparatus (e.g., the test apparatus 1010), according to some embodiments of the present technology. As will be appreciated, acts of the method 4100 need not be performed in the order shown in the flow chart. At act 4102, a sample chamber (e.g., the sample chamber 1016) is arranged in a housing (e.g., the housing 1012) such that the sample chamber may extend through a cavity of the housing, from a first hole in the housing through a second hole in the housing. In some embodiments, the sample chamber may be comprised of a vial portion (e.g., the vial portion 1016b) extending outside of the housing at the first hole and a tube portion (e.g., the tube portion 1016a) located between the first and second holes in the housing. At act 4104, a first seal may be installed at the first hole, to provide a leak-tight seal between the housing and an external surface of the vial portion of the sample chamber. At act 4106, a second seal may be installed at the second hole, to provide a leak-tight seal between the housing and an external surface of the tube portion of the sample chamber. In some embodiments, the first and seals may be rotary seals that enable the sample chamber to be rotated relative to the housing.

FIG. 4C shows a flow chart for a method 4200 for manufacturing a diagnostic test apparatus (e.g., the test apparatus 1010), according to some embodiments of the present technology. At act 4202, a lyophilized reagent (e.g., the reagent 1130) is inserted in a sample chamber (e.g., the sample chamber 1016) of the test apparatus. At act 4204, a blister compartment (e.g., the blister compartment 1042) containing buffer fluid may be added to a vial portion (e.g., the vial portion 1016b) of the sample compartment. A breakable seal may separate the buffer fluid from a hole in the vial portion. At act 4206, a blister compartment (e.g., the blister compartment 1020) containing diluent fluid may be added to a housing cavity (e.g., the housing cavity 1028) of the test apparatus. At act 4208, a test vehicle (e.g., the LFA strip 1030) may be added to the housing cavity.

FIG. 4D shows a flow chart for a method 4300 for manufacturing the diagnostic test kit (e.g., the test kit 1000), according to some embodiments of the present technology. At act 4302, a test apparatus (e.g., the test apparatus 1010) may be provided. For example, the test apparatus may have a housing portion and a sample chamber that are configured to move (e.g., rotate) relative to each other to rupture a blister compartment (e.g., the blister compartment 1020) in the housing. At act 4304, a sample swab (e.g., the sample swab 1080) may be provided. For example, the sample swab may be configured to provide a sample into the sample chamber and to seal an opening of a sample chamber. At act 4306, the test apparatus and the sample swab may be packaged together as a single package. Optionally, reagent(s) and fluid(s) used in a test procedure may be included in the test apparatus and/or included as part of the single package. Optionally, a heater (e.g., the heater 1110) may be included in the test kit, and may be part of the single package.

It should be understood that embodiments of the test apparatus 1010 may be used with diagnostic tests other than those described herein. Similarly, the diagnostic tests described herein may be used with other apparatuses and are not limited to be used with the embodiments of the test apparatus 1010 described herein to perform rapid diagnostic testing for pathogen(s).

### 3. Test Methodologies

The diagnostic devices described herein may be used to detect whether a test subject is afflicted with a communicable disease by detecting whether a target nucleic-acid sequence corresponding to a pathogen of interest and indicative of the disease is present in a sample obtained from the test subject, which may be a human subject, a non-human animal subject, a plant subject, a fungus subject, or a subject comprised of environmental material (e.g., a soil sample, a dust sample, etc.). The sample may be comprised of, for example, any one or any combination of saliva, blood, feces, urine, and mucus obtained from the test subject, and/or may be cells obtained from the test subject by other means (e.g., by scraping the test subject's skin, by cutting/plucking hairs from the test subject, etc.). Target nucleic-acid sequences and techniques that may be used for their detection are described below.

Target nucleic-acid sequences may be associated with a variety of diseases or disorders. In some embodiments of the present technology, the diagnostic devices described herein may be used to diagnose at least one disease or disorder caused by a pathogen. In some embodiments, the diagnostic devices may be configured to detect a nucleic acid encoding a protein (e.g., a nucleocapsid protein) of SARS-CoV-2, which is the virus that causes COVID-19. In some embodiments, the diagnostic devices may be configured to identify particular strains of a pathogen (e.g., a virus). In some embodiments, a diagnostic device may utilize and be comprised of an assay vehicle (e.g., an LFA strip) comprised of a first test line configured to detect a nucleic-acid sequence of SARS-CoV-2 and a second test line configured to detect a nucleic-acid sequence of a SARS-CoV-2 virus having a D614G mutation (i.e., a mutation of the 614^{th} amino acid from aspartic acid (D) to glycine (G)) in its spike protein. In some embodiments, one or more target nucleic-acid sequences may be associated with a single-nucleotide polymorphism (SNP). In certain cases, the diagnostic devices may be used for rapid genotyping to detect whether a SNP, which may affect medical treatment, is present.

In some embodiments of the present technology, the diagnostic devices described herein may be configured to diagnose two or more diseases or disorders. This may be referred to herein as multiplexed testing. In certain cases, for example, a diagnostic device may utilize and be comprised of an LFA strip comprised of a first test line configured to detect a nucleic-acid sequence of SARS-CoV-2, a second test line configured to detect a nucleic-acid sequence of an influenza virus (e.g., an influenza A virus), and a third line configured to detect a nucleic-acid sequence of another influenza virus (e.g., an influenza B virus) or a nucleic acid sequence of a bacterium.

3.1 Lysis of Samples

According to some embodiments of the present technology, lysis may be performed on a sample by chemical lysis techniques (e.g., exposing the sample to one or more lysis reagents) and/or thermal lysis techniques (e.g., heating the sample). In chemical lysis, lysis may be performed by one or more lysis reagents, discussed below.

According to some embodiments of the present technology, a lysis reagent may be in solid form (e.g., lyophilized, dried, crystallized, air jetted, etc.). For example, a solid lysis reagent may be in the form of a pellet, or capsule, or gelcap, or tablet. In some embodiments, a solid lysis reagent may be included in a caged cap, as described in US 2021/0291177 A1, which is incorporated by reference herein in its entirety. In some embodiments, a lysis reagent may be comprised of one or more additional reagents (e.g., a reagent to reduce or eliminate cross contamination).

According to some embodiments of the present technology, a solid lysis reagent may be shelf stable for a relatively long period of time. In some embodiments, a lysis pellet, or capsule, or gelcap, or tablet may be shelf stable for at least 1 month, at least 3 months, at least 6 months, at least 1 year, at least 5 years, or at least 10 years. In some embodiments, a solid lysis reagent may be thermostabilized and may be stable across a wide range of temperatures. In some embodiments, a lysis pellet, or capsule, or gelcap, or tablet may be stable at a temperature of at least 0° C., at least 10° C., at least 20° C., at least 37° C., at least 65° C., or at least 100° C. As will be appreciated, a solid lysis reagent may be activated before or during use with a sample by contact with a buffer fluid.

As noted above, thermal lysis may be accomplished by applying heat to a sample. According to some embodiments of the present technology, thermal lysis may be performed by applying a lysis heating protocol comprised of heating the sample at one or more temperatures for one or more time periods or durations using any suitable heater (e.g., the heater 1110).

3.2 Nucleic-Acid Amplification

Following lysis, one or more target nucleic acids (e.g., a nucleic acid of a target pathogen) may be amplified, according to some embodiments of the present technology. In some embodiments, DNA may be amplified according to any nucleic-acid amplification method known in the art. For example, nucleic-acid amplification methods that may be employed may include isothermal amplification methods, which include: loop-mediated isothermal amplification (LAMP), recombinase polymerase amplification (RPA), nicking enzyme amplification reaction (NEAR), thermophilic helicase dependent amplification (tHDA), nucleic acid sequence-based amplification (NASBA), strand displacement amplification (SDA), isothermal multiple displacement amplification (IMDA), rolling circle amplification (RCA), transcription mediated amplification (TMA), signal mediated amplification of RNA technology (SMART), single primer isothermal amplification (SPIA), circular helicase-dependent amplification (cHDA), whole genome amplification (WGA), and CRISPR-related amplification, such as CRISPR-Cas9-triggered nicking endonuclease-mediated strand displacement amplification (CRISDA). In some embodiments, an isothermal amplification method that may be performed in a test procedure may be comprised of applying heat to a sample. For example, heat may be applied to a sample fluid containing the sample. In some embodiments, the isothermal amplification method may be comprised of applying an amplification heating protocol, which may be comprised of heating the sample at one or more temperatures for one or more time periods using any appropriate heater (e.g., the heater 1110).

In embodiments where a target pathogen may have RNA as its genetic material, the target pathogen's RNA may need to be reverse transcribed to DNA prior to amplification.

3.3 Molecular Switches

As described herein, a sample may undergo lysis and amplification prior to detection of a target nucleic-acid sequence. Reagents associated with lysis and/or amplification may be in solid form (e.g., lyophilized, dried, crystallized, air jetted, etc.). According to some embodiments of the present technology, one or more (and, in some cases, all) of the reagents necessary for lysis and/or amplification may be present in a single pellet, capsule, gelcap, or tablet. In some embodiments, the pellet, capsule, gelcap, or tablet may be comprised of two or more enzymes, and it may be necessary for the enzymes to be activated in a particular order. Therefore, in some embodiments, the enzyme-containing tablet, pellet, capsule, or gelcap may further be comprised of one or more molecular switches.

Molecular switches, as used or described herein, may be molecules that, in response to certain conditions, reversibly switch between two or more stable states. According to some embodiments of the present technology, a condition that causes a molecular switch to change its configuration may be associated with any one or any combination of: pH, light, temperature, an electric current, microenvironment, and presence of ions and/or other ligands. In some embodiments, the condition may be heat. In some embodiments, the molecular switches may be comprised of aptamers. Aptamers may refer generally to oligonucleotides or peptides that may bind to specific target molecules (e.g., the enzymes described herein). The aptamers, upon exposure to heat or other conditions, may dissociate from the enzymes. With use of molecular switches, one or more of the processes described herein (e.g., lysis, decontamination, reverse transcription, amplification, etc.) may be performed in a single test tube with a single enzymatic tablet, pellet, capsule, or gelcap.

3.4 CRISPR/Cas Techniques

According to some embodiments of the present technology, CRISPR/Cas detection techniques may be used to detect a target nucleic-acid sequence. For example, one or more CRISPR/Cas detection reagents may be included on an LFA strip. CRISPR generally may refer to Clustered Regularly Interspaced Short Palindromic Repeats, and Cas generally may refer to a particular family of proteins. In some embodiments, a CRISPR/Cas detection platform or technique may be combined with an isothermal amplification method to create a single-step reaction (Joung et al., “Point-of-care testing for COVID-19 using SHERLOCK diagnostics,” 2020). For example, amplification and CRISPR detection may be performed using reagents having compatible chemistries (e.g., reagents that do not interact detrimentally with one another and are sufficiently active to perform amplification and detection). In some embodiments, CRISPR/Cas detection may be combined with LAMP.

### 4. Reagents

According to some embodiments of the present technology, the diagnostic apparatus (e.g., the test apparatus 1010) described herein may include and/or utilize reagents (e.g., lysis reagents, nucleic-acid amplification reagents, CRISPR/Cas detection reagents, and the like) in various test procedures of a diagnostic test. In some embodiments, one or more of the reagents may be contained within the diagnostic apparatus (e.g., in the internal cavity 1018 of the sample chamber 1016). In some embodiments, one or more of the reagents may be provided separately (e.g., in one or more caged caps, in one or more separate vials, etc.). For example, the test apparatus 1010 may be comprised of one or more caged caps comprising one or more lysing reagents and/or one or more amplification reagents that may be added to the sample chamber 1016.

According to some embodiments of the present technology, at least one (and, in some instances, each) of the reagents used in a diagnostic test may be in liquid form (e.g., in solution). In some embodiments, at least one (and, in some instances, each) of the reagents used in a diagnostic test may be in solid form (e.g., lyophilized, dried, crystallized, air jetted, and the like) and may be activated with buffer fluids prior to or during use.

4.1 Lysing Reagents

According to some embodiments of the present technology, the reagents may be comprised of one or more lysis reagents. A lysis reagent may refer generally to a reagent that promotes cell lysis either alone or in combination with one or more other reagents and/or one or more conditions (e.g., heating). In some embodiments, the lysis reagents may be comprised of one or more enzymes. Non-limiting examples of suitable enzymes may include lysozyme, lysostaphin, zymolase, cellulose, protease, and glycanase. In some embodiments, the lysis reagent(s) may be comprised of one or more detergents. Non-limiting examples of suitable detergents may include sodium dodecyl sulphate (SDS), Tween (e.g., Tween 20, Tween 80), 3-[(3-cholamidopropyl)dimethylammonio]-1-propanesulfonate (CHAPS), 3-[(3-cholamidopropyl)dimethylammonio]-2-hydroxy-1-propanesulfonate (CHAPSO), Triton X-100, and NP-40. In some embodiments, the lysis reagents may be comprised of an RNase inhibitor (e.g., a murine RNase inhibitor). In some embodiments, a concentration of the RNase inhibitor may be is at least 0.1 U/μL, at least 1.0 U/μL, or at least 2.0 U/μL. In some embodiments, the concentration of the RNase inhibitor may be in a range from 0.1 U/μL to 0.5 U/μL, 0.1 U/μL to 1.5 U/μL, or 1.0 U/μL to 2.0 U/μL. In some embodiments, the lysis reagents may comprise Tween (e.g., Tween 20, Tween 80).

4.2 Contamination-Prevention Reagents

According to some embodiments of the present technology, the reagents may be comprised of at least one reagent that works to reduce or eliminate potential carryover contamination from prior tests (e.g., prior tests conducted with a common apparatus and/or in a same area). In some embodiments, the reagents may be comprised of thermolabile uracil DNA glycosylase (UDG). In some embodiments, UDG may prevent carryover contamination from prior tests by degrading products that have already been amplified (i.e., amplicons) while leaving unamplified samples untouched and ready for amplification. In some embodiments, a concentration of UDG may be at least 0.01 U/μL, at least 0.03 U/μL, or at least 0.05 U/μL. In some embodiments, the concentration of UDG may be in a range from 0.01 U/μL to 0.02 U/μL or 0.01 U/μL to 0.04 U/μL.

4.3 Reverse Transcription Reagents

According to some embodiments of the present technology, the reagents may be comprised of one or more reverse transcription reagents. As noted above, a target pathogen may have RNA as its genetic material, which may need to be reverse transcribed to DNA prior to amplification. In some embodiments, the reverse transcription reagents may facilitate such reverse transcription. In some embodiments, the reverse transcription reagents may be comprised of a reverse transcriptase, a DNA-dependent polymerase, and/or a ribonuclease (RNase). A reverse transcriptase may refer generally to an enzyme that transcribes RNA to complementary DNA (cDNA) by polymerizing deoxyribonucleotide triphosphates (dNTPs). An RNase may refer generally to an enzyme that catalyzes the degradation of RNA. In some embodiments, an RNase may be used to digest RNA from an RNA-DNA hybrid.

4.4 Nucleic-Acid Amplification Reagents

According to some embodiments of the present technology, the reagents may comprise one or more nucleic-acid amplification reagents. In some embodiments, the nucleic-acid amplification reagents may comprise LAMP reagents, RPA reagents, and NEAR reagents, known in the art. In some embodiments, an enzyme (e.g., Bsm DNA polymerase) may serve as an amplification reagent.

4.5 Reagent Stability Enhancers

According to some embodiments of the present technology, the reagents may comprise one or more additives that may enhance reagent stability (e.g., protein stability). Non-limiting examples of suitable additives may include trehalose, polyethylene glycol (PEG), polyvinyl alcohol (PVA), and glycerol.

4.6 Buffers

According to some embodiments of the present technology, the reagents may comprise one or more reaction buffers. Non-limiting examples of suitable buffers may include phosphate-buffered saline (PBS) and Tris. In some embodiments, the buffers may be buffer fluids. In some embodiments, the buffers may have a relatively neutral pH. In some embodiments, the buffers may have a pH in a range from 5.0 to 7.0, 6.0 to 8.0, 7.0 to 9.0, or 8.0 to 9.0. In some embodiments, the buffers may comprise one or more salts. Non-limiting examples of suitable salts may include magnesium acetate tetrahydrate, potassium acetate, and potassium chloride. In some embodiments, the buffers may comprise Tween (e.g., Tween 20, Tween 80). In some embodiments, the buffers may comprise an RNase inhibitor. In some embodiments, Tween and/or an RNase inhibitor may facilitate cell lysis. In a particular, non-limiting embodiment of the present technology, the buffers may comprise 25 mM Tris buffer, 5% (w/v) poly(ethylene glycol) 35,000 kDa, 14 mM magnesium acetate tetrahydrate, 100 mM potassium acetate, and greater than 85% volume nuclease free water.

### 5. Detection Devices

As noted above, according to some embodiments of the present technology, LFA strips (e.g., the LFA strip 1030) may be used as assay vehicles to test for whether a target nucleic-acid sequence, corresponding to a pathogen of interest, is present in a sample obtained from a user. In some embodiments, the target nucleic acid-acid sequence may be amplified (i.e., amplicons) prior to detection via an LFA strip. In some embodiments, an LFA strip may provide results that may be read or interpreted in a non-clinical setting by a lay person (e.g., a person not trained in laboratory procedures). LFA strips may be comprised of reagents or substances for indicating the presence (or absence) of a target nucleic-acid sequence. In some embodiments, an LFA strip may be configured to detect two or more different target nucleic-acid sequences.

According to some embodiments of the present technology, an LFA strip useable with the diagnostic test apparatuses described herein may be comprised of one or more fluid-transporting layers, which may be comprised of one or more absorbent materials that allow a fluidic sample to move from one end of the LFA strip (e.g., an intake end) to an opposite end of the LFA strip. In some embodiments, fluid movement may be via wicking or capillary action. Non-limiting examples of suitable materials may include polyethersulfone, cellulose, polycarbonate, nitrocellulose, sintered polyethylene, and glass fibers.

According to some embodiments of the present technology, an LFA strip may be comprised of a plurality of sub-regions. In some embodiments, the fluidic sample may be introduced to a first sub-region (e.g., a region in contact with a sample pad) and may subsequently flow through a second sub-region (e.g., a particle conjugate pad) comprised of a plurality of labeled particles. In some embodiments, the particles may be comprised of gold nanoparticles (e.g., colloidal gold nanoparticles). The particles may be labeled with any suitable label. Non-limiting examples of suitable labels include biotin, streptavidin, fluorescein isothiocyanate (FITC), fluorescein amidite (FAM), fluorescein, and digoxigenin (DIG). In some embodiments, as an amplicon-containing fluidic sample flows through the second sub-region, a labeled nanoparticle may bind to a label of an amplicon, thereby forming a particle-amplicon conjugate. In some embodiments, the fluidic sample may subsequently flow through a third sub-region comprised of one or more test lines. In some embodiments, a first test line may be comprised of a capture reagent (e.g., an immobilized antibody) configured to detect a first target nucleic-acid sequence. In some embodiments, a particle-amplicon conjugate may be captured by one or more capture reagents (e.g., immobilized antibodies), and an opaque marking may appear on the first test line. In some embodiments, the LFA strip may comprise one or more additional test lines configured to detect one or more different target nucleic-acid sequences. In some embodiments, the third sub-region of the LFA strip may further comprise one or more control lines. For example, a control line may be a human (or animal) nucleic-acid control line configured to detect a nucleic acid (e.g., RNase P) that is generally present in all humans (or animals). The control line may be used to confirm whether a human (or animal) sample was successfully collected, nucleic-acid sequences from the sample were amplified, and the amplicons were transported through the LFA strip successfully.

According to some embodiments of the present technology, a diagnostic device may be comprised of two or more LFA strips arranged in parallel, such that a sample fluid may flow in each LFA strip independently of the other LFA strip(s).

### 6. Test Kits

According to some embodiments of the present technology, the diagnostic test apparatuses described herein may be part of a test kit useable by a lay person, i.e., a person who is not trained in medical and/or laboratory techniques or procedures. The test kit may be a stand-alone test kit that does not require the use of additional laboratory equipment to perform a diagnostic test. In some embodiments, the test kit may be comprised of a swab device (e.g., the sample swab 1080) and a diagnostic apparatus (e.g., the test apparatus 1010). One or more reagents necessary for the diagnostic test may be provided in the diagnostic apparatus (e.g., in the sample chamber 1016) or may be provided in a reagent carrier (e.g., a caged cap) to be added by a user during a test procedure.

6.1 Heater

According to some embodiments of the present technology, the test apparatus 1010 may be used with the heater 1120, as discussed above. In some embodiments, the heater 1110 may be provided in compact form. The heater 1110 may be comprised of a battery-powered heat source, a USB-powered heat source (e.g., a source able to be powered via a USB terminal of a computer or other electronic device), of a heat source that may be powered conventionally (e.g., via a wall outlet of a home). In some instances, the heater 1110 may be a printed circuit board (PCB) heater. For example, the PCB heater may be part of a protective case in which the test apparatus 1010 may be transported and/or stored. The PCB heater, in some embodiments, may be comprised of a bonded PCB with a microcontroller, thermistors, and/or resistive heaters, and may be configured to receive the vial portion of 1016b of the sample chamber 1016. Heating by the PCB heater may not occur until the PCB heater is powered on.

In some embodiments of the present technology, the heater 1110 may be comprised of a battery-powered heater, a USB-powered heater, or a heater that may be powered conventionally (e.g., via a wall outlet of a home). In certain embodiments, the heater 1110 may be contained within a thermally insulated housing to ensure user safety. In certain instances, the heater 1110 may be an off-the-shelf consumer-grade device. In some embodiments, the heater 1110 may be a thermocycler or other specialized laboratory equipment known in the art and configured to receive the vial portion 1016b.

In some embodiments of the present technology, the heater 1110 may be configured to heat the vial portion 1016b at a temperature of at least 37° C., at least 65° C., or at least 90° C. In some embodiments, the heater 1110 may be configured to heat the vial portion 1016b at a temperature for at least 5 minutes, at least 30 minutes, or at least 90 minutes.

In some embodiments of the present technology, the heater 1110 may be comprised of at least two temperature zones. In certain instances, for example, the heater 1110 may include a heating element (e.g., a resistive device) connected to a microcontroller that may switch between two temperature zones. In some embodiments, the heater 1110 may be configured to heat the vial portion 1016b to a plurality of temperatures for a plurality of time periods. In some embodiments, for example, the heater 1110 may be configured to heat the vial portion 1016b at a first temperature for a first period of time and at a second temperature for a second period of time. In some embodiments, the heater 1110 may be pre-programmed with one or more protocol(s). In some embodiments, for example, the heater 1110 may be pre-programmed with a lysis heating protocol and/or an amplification heating protocol. A lysis heating protocol may generally refer to a set of one or more temperatures and one or more time period(s) that facilitate lysis of the sample. An amplification heating protocol may generally refer to a set of one or more temperature(s) and one or more time period(s) that facilitate nucleic-acid amplification. In some embodiments, the heater 1110 may comprise an auto-start mechanism that corresponds to the temperature profile needed for lysis and/or amplification. For example, when the vial portion 1016b is detected to be inserted in the aperture 1110a of the heater 1110, the heater 1110 may automatically run a lysis and/or amplification heating protocol. In some embodiments, the heater 1110 may be controlled via a mobile application on, e.g., a smartphone.

6.2 Instructions & Software

According to some embodiments of the present technology, a test kit may be comprised instructions associated with sample collection and/or operation of a diagnostic apparatus (e.g., the test apparatus 1010). For example, the instructions may be comprised of directions for handling a swab device (e.g., the sample swab 1080) to obtain a sample from a subject as well as directions for providing a collected sample to a diagnostic apparatus (or a component thereof) for further processing. The instructions may be provided in any form readable by a user. For example, the instructions may be written or published, verbal, audible (e.g., telephonic), digital, optical, visual (e.g., videotape, DVD, etc.), and/or provided via electronic communications (including Internet or web-based communications). In some embodiments, the instructions may combine graphical information with textual information. In some embodiments, the instructions may be provided as part of a software-based application.

According to some embodiments of the present technology, the instructions may be provided as part of a software-based application that may be downloaded to a smartphone or other type of portable electronic device, and contents of the downloaded application may guide a user through steps to use a diagnostic apparatus and/or to perform test procedures of a diagnostic test. In some embodiments, the instructions may instruct a user when to add certain reagents and how to do so.

According to some embodiments of the present technology, a software-based application may be connected (e.g., via a wired or wireless connection) a diagnostic device to control the diagnostic device or components thereof and/or to read and analyze test results. In some embodiments, the application may be configured to process an image of an LFA strip captured by an imaging device (e.g., a smartphone camera, etc.) and to evaluate the image to provide a positive or negative test result for each of one or more test lines on the LFA strip.

It should be understood that the features and details described above may be used, separately or together in any combination, in any of the embodiments discussed herein.

Some aspects of the present technology may be embodied as one or more methods. Acts performed as part of a method may be ordered in any suitable way. Accordingly, embodiments may be constructed in which acts may be performed in an order different than described or illustrated, which may include performing some acts simultaneously, even though they may be shown or described as sequential acts in illustrative embodiments.

Further, test kits of the present technology may be comprised of any combination of component(s) and/or apparatus(es) described herein and are not limited to any particular combination(s) described herein.

Furthermore, although advantages of the present technology may be indicated, it should be appreciated that not every embodiment of the present technology may include every described advantage. Some embodiments may not implement any feature described as advantageous herein. Accordingly, the foregoing descriptions and attached drawings are by way of example only.

Variations and modifications of the disclosed embodiments are possible and are within the scope of the present technology. For example, various aspects of the present technology may be used alone, in combination, or in a variety of arrangements not specifically discussed in the embodiments described above, and therefore they are not limited in application to the details and arrangements of components set forth in the foregoing description or illustrated in the drawings. Aspects described in one embodiment may be combined in any manner with aspects described in other embodiments.

Any use of ordinal terms such as “first,” “second,” “third,” etc., in the description and the claims to modify an element does not by itself connote any priority, precedence, or order of one element over another, or the temporal order in which acts of a method are performed, but is or are used merely as labels to distinguish one element or act having a certain name from another element or act having a same name (but for use of the ordinal term) to distinguish the elements or acts.

The indefinite articles “a” and “an,” as used herein in the specification and in the claims, unless clearly indicated to the contrary, should be understood to mean “at least one.”

Any use herein, in the specification and in the claims, of the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements.

Any use herein, in the specification and in the claims, of the phrase “equal” or “the same” in reference to two values (e.g., distances, widths, etc.) should be understood to mean that two values are the same within manufacturing tolerances. Thus, two values being equal, or the same, may mean that the two values are different from one another by ±5%.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. As used herein in the specification and in the claims, the term “or” should be understood to have the same meaning as “and/or” as defined above.

The terms “approximately” and “about” if used herein may be construed to mean within ±20% of a target value in some embodiments, within ±10% of a target value in some embodiments, within ±5% of a target value in some embodiments, and within ±2% of a target value in some embodiments. The terms “approximately” and “about” may equal the target value.

The term “substantially” if used herein may be construed to mean within 95% of a target value in some embodiments, within 98% of a target value in some embodiments, within 99% of a target value in some embodiments, and within 99.5% of a target value in some embodiments. In some embodiments, the term “substantially” may equal 100% of the target value.

What is claimed is:

1. A rapid diagnostic test apparatus, comprising:
a housing;
a sample chamber movably coupled to the housing; and
a housing blister compartment arranged in the housing and configured to rupture upon a movement of the housing relative to the sample chamber.

2. The apparatus of claim 1, wherein the sample chamber is comprised of a vial portion.

3. The apparatus of claim 2, further comprising:
a vial blister compartment sealing an opening of the vial portion.

4. The apparatus of claim 3, wherein a tube portion of the sample chamber is disposed in the housing, and the vial portion extends from the housing.

5. The apparatus of claim 1, wherein:
the sample chamber is rotatably coupled to the housing, and
the housing blister compartment is configured to rupture when the housing is rotated relative to the sample chamber from a first position to a second position.

6. The apparatus of claim 5, wherein:
the housing blister compartment contains a fluid, and
in the second position, the fluid is released into the housing.

7. A rapid diagnostic test apparatus, comprising:
a housing;
a sample chamber comprised of a vial coupled to the housing; and
a first blister compartment sealing an opening of the vial and configured to rupture upon application of a rupture force.

8. The apparatus of claim 7, wherein application of the rupture force to the first blister compartment causes the first blister compartment to be in fluid communication with an internal cavity of the sample chamber.

9. The apparatus of claim 7, wherein the vial extends from a first opening in the housing.

10. The apparatus of claim 9, wherein the sample chamber is comprised of:
the vial, and
a tube housed in a cavity of the housing and in fluid communication with the vial.

11. The apparatus of claim 10, wherein:
the sample chamber is rotatable relative to the housing from a rest position to a test position, and
the housing is comprised of:
a first rotary seal located at the first opening in the housing and configured to provide a leak-tight seal between the housing and the external surface of the vial of the sample chamber, and
a second rotary seal located at a second opening in the housing and configured to provide a leak-tight seal between the housing and an external surface of the tube of the sample chamber.

12. The apparatus of claim 11, wherein the internal cavity of the sample chamber is configured to receive a sample swab.

13. The apparatus of claim 12, further comprising:
a second blister compartment disposed in the cavity of the housing,
wherein the second blister compartment is configured to rupture upon application of a second rupture force.

14. The apparatus claim 13, wherein the tube of the sample chamber is comprised of:
at least one rib extending from an outer surface of the tube, and
a plurality of conduits connecting the cavity of the housing to the internal cavity of the sample chamber.

15. The apparatus of claim 14, wherein the housing is comprised of a plurality of blockers disposed in the cavity, the blockers being configured such that:
when the sample chamber is in the rest position relative to the housing, the blockers block the conduits of the tube of the sample chamber such that the cavity of the housing is not in fluid communication with the internal cavity of the sample chamber, and
when the sample chamber is in the test position relative to the housing, the blockers do not block the conduits of the tube of the sample chamber such that the cavity of the housing is in fluid communication with internal cavity of the sample chamber.

16. The apparatus of claim 15, wherein, when the housing is rotated relative to the sample chamber to the test position, a surface of the tube of the sample chamber contacts the second blister compartment to rupture the second blister compartment.

17. The apparatus of claim 15, further comprising:
a linear-flow assay (LFA) strip disposed in the cavity of the housing,
wherein the housing is comprised of a window through which a test region of the LFA strip is visible.

18. A rapid diagnostic test kit, comprising:
a rapid diagnostic test apparatus comprised of:
a housing,
a sample chamber movably coupled to the housing, and
a housing blister compartment arranged in the housing and configured to rupture; and
a reagent configured to interact with a sample during a test procedure of the test apparatus.

19. The test kit of claim 18, further comprising:
a sample swab configured to obtain a sample from a subject and to deliver the sample into the sample chamber, or
a heater configured to heat the sample chamber, or
both the sample swab and the heater.

20. The test kit of claim 18, wherein:
a housing blister compartment is configured to rupture when the housing is rotated relative to the sample chamber from a first position to a second position, and
the housing blister compartment contains a fluid configured to activate the reagent.
